(19) United States
(12) Patent Application Publication
(10) Pub. No.: US 2013/0072720 A1
(43) Pub. Date: Mar. 21, 2013

(54) PHARMACEUTICAL COMPOSITION FOR TREATMENT OF OSTEOARTHRITIS

(71) Applicants: HSU-SHAN HUANG, Taipei City (TW); JENN-HAUNG LAI, Taipei City (TW); LING-JUN HO, Taipei City (TW); FENG-CHENG LIU, Taipei City (TW)

(72) Inventors: HSU-SHAN HUANG, Taipei City (TW); JENN-HAUNG LAI, Taipei City (TW); LING-JUN HO, Taipei City (TW); FENG-CHENG LIU, Taipei City (TW)

(73) Assignee: NATIONAL DEFENSE MEDICAL CENTER, Taipei City (TW)

(21) Appl. No.: 13/337,661

(22) Filed: Dec. 27, 2011

(30) Foreign Application Priority Data
Sep. 20, 2011 (TW) 100133846

(51) Int. Cl. C07C 235/56

(52) U.S. Cl. 564/179

(57) ABSTRACT

A pharmaceutical composition including 2-hydroxy-N—[3-(trifluoromethyl)phenyl]benzamide and used for treatment of osteoarthritis is revealed. The pharmaceutical composition inhibits tumor necrosis factor (TNF) induced interferon regulatory factor (IRF). The activated IRF stimulates chondrocytes to secret matrix metalloproteinases, inducible nitric oxide synthase (iNOS), aggrecanases, etc. This leads to loss of collagen II and further causes degradation of proteoglycan. By suppression of signaling pathways of interferon regulatory factor, symptoms are relieved and osteoarthritis is treated.

### BACKGROUND OF THE INVENTION

1. Field of the Invention

The present invention relates to a pharmaceutical composition, especially to a pharmaceutical composition used as potential drug candidates for treatment of osteoarthritis.

2. Descriptions of Related Art

Osteoarthritis is the most common joint disorder, also known as degenerative arthritis. The knee joint is the largest joint in the body and is one of the most easy to get degenerated and damaged joints. The knee joint is covered by a layer of cartilage that is composed of chondrocytes, collagen and proteoglycans secreted by chondrocytes, and water. The cartilage is like a pad between the ends of bones which form joints, acting as a buffer. The degenerative arthritis of the knee joint is likely to get progressively worse as people age. The degenerative arthritis is the result of long-term stresses on a joint. After a long time, the cartilage at a surface of the knee joint is damaged. Moreover, synovial fluid in the knee joint capsule is reduced. These lead to clinical syndromes of the knee joint such as pain, swelling, disability, having difficulty in squatting and sitting.

The most frequently prescribed medications for arthritis pain are NSAIDs (non-steroidal anti-inflammatory drugs). In the past, steroids were most commonly used in the treatment of degenerative arthritis besides physical therapy that decreases pain. However, the steroids have a plenty of common side effects such as upper gastrointestinal bleeding, thinning of the skin, osteoporosis, slower wound healing, etc.

For people who already have diabetes, high blood pressure, etc., the disease may get worse. Thus most doctors stop prescribing these drugs.

In recent years, glucosamine has been used to treat patients with degenerative arthritis. Glucosamine has also been shown to spur chondrocytes to produce more collagen and proteoglycans, and it also normalizes cartilage metabolism, which helps to keep cartilage from breaking down and further rebuilds the damaged cartilage. Another way is to use liquid joint supplements such as sodium hyaluronate that is administered through injections into the degenerative knee joints, increasing the viscosity of the synovial fluid. The supplement is covered over the cartilage, helping to hydrate and lubricate joints and preventing friction and joint degeneration.

However, glucosamine also has side effects including gastrointestinal discomfort, nausea, diarrhea, etc. The glucosamine acts on the increasing production of cartilage matrix, without slowing down the degeneration of cartilage matrix. As to the sodium hyaluronate, it's more difficult to secure material and the injection is an invasive procedure. Moreover, normal cartilage metabolism is a highly regulated balance between synthesis and degradation of various matrix components. The balance helps maintain healthy cartilage and smooth movement of joints. For osteoarthritis patients, the balance between synthesis and degradation is disturbed as a result of an exposure of chondrocytes to various cytokines and growth factors. Thus the cartilage degradation is faster than the synthesis of matrix components. Within the balance of the cartilage, the relief of degeneration and damage of cartilage matrix is as important as the increasing of synthesis of cartilage matrix components.

The chondrocytes are simulated and various cytokines such as interleukins, tumor necrosis factors, etc are secreted when the joints are subject to high level of stress (such as injuries). The propagation of cytokines further induces secretion of metalloproteinases (MMP). The MMPs destroy collagen fibers and damages the cartilage matrix components. Thus most of new drugs that suppress destruction of cartilage matrix focus on blocking Interleukin-1 receptors or suppressing MMP activity so as to protect articular cartilage.

### SUMMARY OF THE INVENTION

Therefore it is a primary object of the present invention to provide a pharmaceutical composition for treatment of osteoarthritis that suppresses tumor necrosis factor (TNF) induced interferon regulatory factor (IRF).

It is another object of the present invention to provide a pharmaceutical composition for treatment of osteoarthritis that prevents TNF (tumor necrosis factor)-mediated collagen loss and proteoglycan loss.

It is a further object of the present invention to provide a pharmaceutical composition for treatment of osteoarthritis that relieve symptoms of osteoarthritis and slows down degradation.

It is a further object of the present invention to provide a pharmaceutical composition for treatment of osteoarthritis whose cytotoxicity is undetectable and hence have more applications.

In order to achieve the above objects, a pharmaceutical composition for treatment of osteoarthritis that suppresses tumor necrosis factor (TNF) induced interferon regulatory factor (IRF) according to the present invention is provided. The activation of IRF stimulates chondrocytes to secret matrix metalloproteinases, inducible nitric oxide synthase (iNOS), aggrecanases, etc and this causes loss of collagen II and further the degradation of proteoglycan. Thus the symptoms of osteoarthritis are relieved and the treatment is effective by suppression of signaling pathways of IRF.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a chemical structure of an embodiment according to the present invention;

FIG. 2 shows a synthesis reaction of an embodiment according to the present invention;

FIG. 3 shows suppression of inducible nitric oxide synthase (iNOS) by an embodiment according to the present invention;

FIG. 4 shows suppression effect of an embodiment on matrix metalloproteinase-1 (MMP-1) according to the present invention;

FIG. 5 shows suppression effect of an embodiment on metalloproteinase-3 (MMP-3) according to the present invention;

FIG. 6 shows suppression effect of an embodiment on matrix metalloproteinase-13 (MMP-13) according to the present invention;

FIG. 7 shows suppression effect of an embodiment on Aggrecanase-1 (ADAMTS-4) according to the present invention;

FIG. 8 shows suppression effect of an embodiment on Aggrecanase-2 (ADAMTS-5) according to the present invention;

FIG. 9A shows loss of collagen II in the absence of an embodiment according to the present invention;

FIG. 9B shows loss prevention effect of an embodiment on collagen II according to the present invention;

FIG. 10 shows loss prevention effect of an embodiment on proteoglycan according to the present invention.

### DETAILED DESCRIPTION OF THE PREFERRED EMBODIMENT

In prior arts related to treatment of arthritis, most of them increase production of cartilage matrix, instead of suppressing the degeneration and damage of cartilage matrix. The present invention provides a pharmaceutical composition for treatment of osteoarthritis that focuses on suppression of degeneration and damage of cartilage matrix.

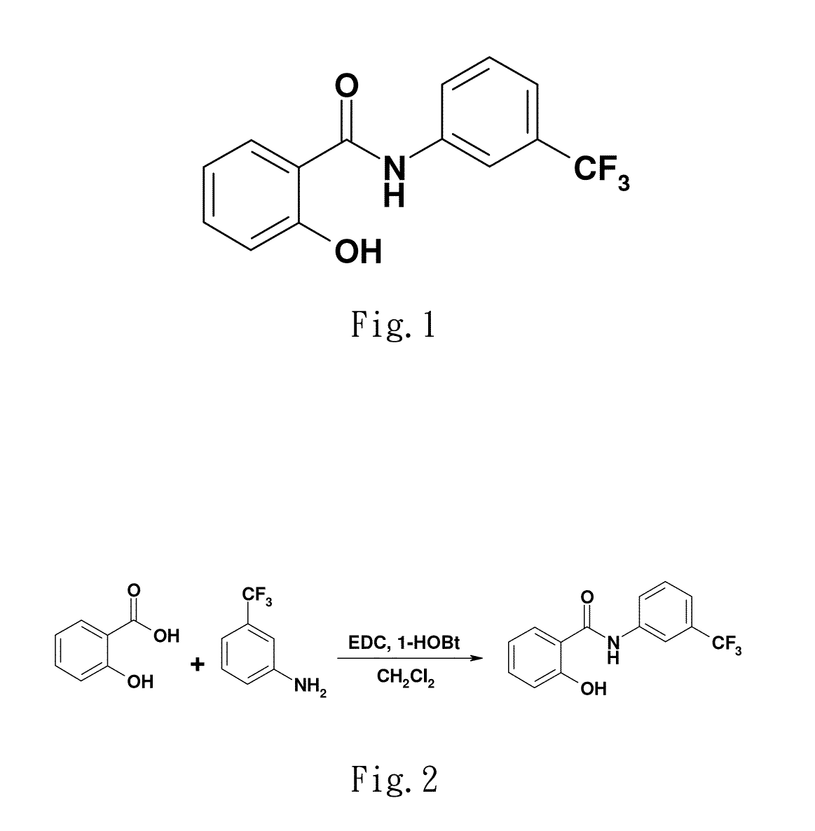

Refer to FIG. 1, a chemical structure of an embodiment according to the present invention is revealed. As shown in figure, the compound is 2-hydroxy-N-[3-(trifluoromethyl) phenyl]benzamide (hereafter referred to as HS—Cf).

Refer to FIG. 2, a method for the synthesis of HS—Cf is revealed. As shown in the figure, dissolve 0.276 g (2 mmol) salicylic acid into 10 ml dichloromethane. Then add 0.270 g (2 mmol) 1H-Benzotriazol-1-ol hydrate and 0.270 g (2 mmol) Ethylene dichloride (EDC) into the solution. Next 0.354 ml (2.2 mmol) 3-trifluoromethylaniline is further added into and reacted with the solution for 72 hours. The reacted solution is processed by vacuum concentration, extracted by water and methylene chloride, dehydrated by magnesium sulfate, and vacuum concentration again to get crude product. Next the crude product is dissolved in hot ethanol and the solid is recrystallized so as to obtain white compound C₁₄H₁₀F₃NO₂ (HS—Cf), which is a principal component of the pharmaceutical composition of the present invention.

H-NMR spectrum (300 MHz, DMSO) δ(ppm) of HS—Cf (C₁₄H₁₀F₃NO₂) is: 6.835-7.004 (m, 2H, H-1,3), 7.374-7.536 (m, 2H, H-6,7), 7.551-7.623 (m, 1H, H-2,5), 7.891-7.984 (m, 2H, H-4), 8.212 (s, 1H, H-8), 10.590 (s, 1H, NH), 11.519 (s, 1H, OH).

The activity of TNF-α-induced IRF-1 activity is suppressed by the synthetic HS—Cf so as to prevent cartilage damage and destruction and relieve symptoms of osteoarthritis.

As to IRF-1, it activates matrix metalloproteinases, inducible nitric oxide synthase (iNOS), and aggrecanases. The matrix metalloproteinases include matrix metalloproteinase-1, matrix metalloproteinase-3 and matrix metalloproteinase-13 while the aggrecanases include aggrecanase-1 (A Disintegrin and Metalloproteinase with Thrombospondin motifs-4, ADAMTS-4) and aggrecanase-2 (A Disintegrin and Metalloproteinase with Thrombospondin motifs-5, ADAMTS-5). By suppressing IRF-1, the activity of above enzymes is down-regulated so as to relieve symptoms of osteoarthritis.

Moreover, HS—Cf further prevents TNF (tumor necrosis factor)-mediated collagen loss and proteoglycan (aggrecan) loss.

The following embodiments show suppression effect of HS—Cf on those factors involved in signaling pathways of TNF-α-induced IRF-1 expression and prevention effect of HS—Cf on TNF-mediated collagen loss and proteoglycan loss. All porcine chondrocytes used are obtained from the hind leg joints of pigs.

Suppression of Inducible Nitric Oxide Synthase (iNOS)

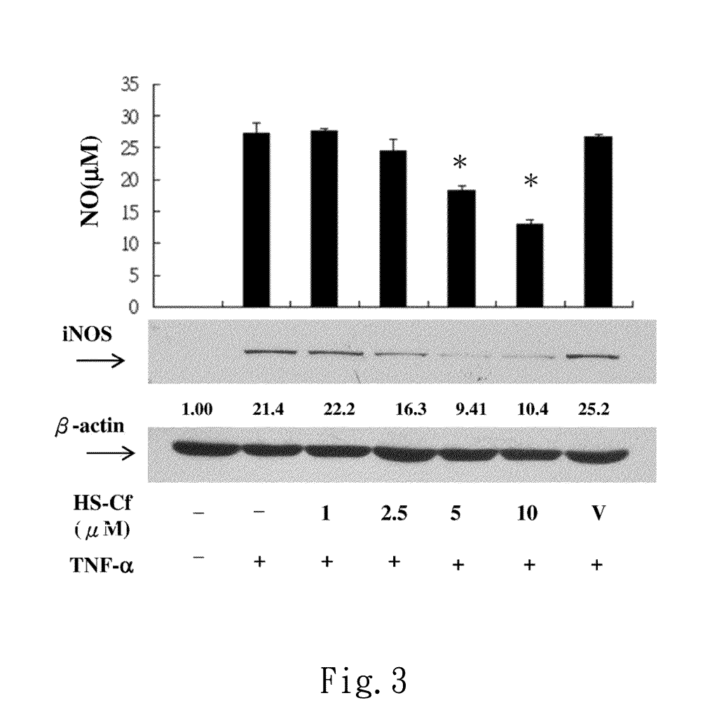

Refer to FIG. 3, it shows that inducible nitric oxide synthase (iNOS) expression is suppressed by HS—Cf of the present invention. NO (nitric oxide) is generated by TNF-α-induced iNOS so that iNOS expression is correlated with NO production. HS—Cf in different concentrations are delivered to chondrocytes stimulated by TNF-α (5 ng/ml). After 48 hours, HS—Cf significantly decreases iNOS expression at higher concentration and NO production is also reduced along with the increasing concentration of HS—Cf.

### Suppression of Matrix Metalloproteinase-1 (MMP-1)

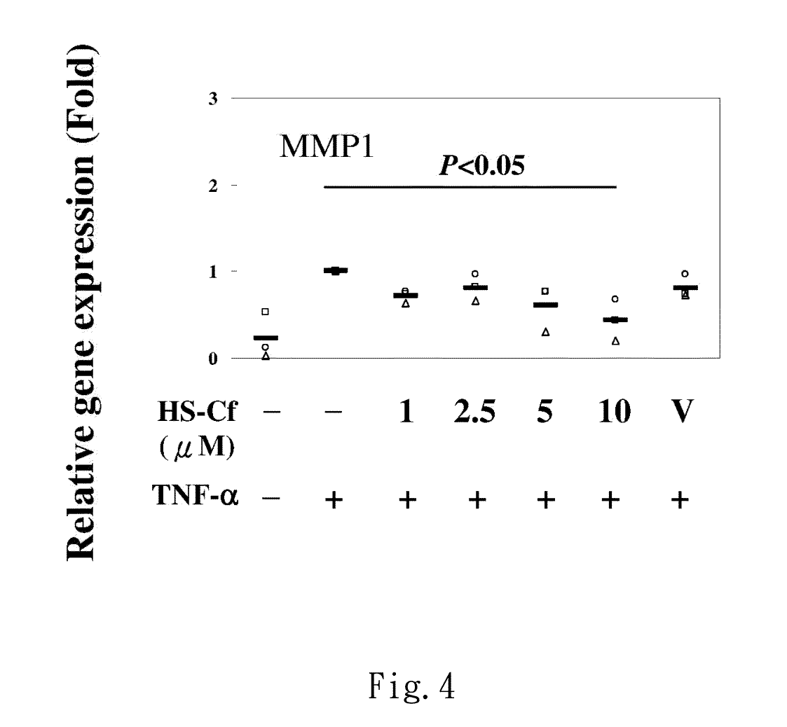

Refer to FIG. 4, it shows that matrix metalloproteinase-1 (MMP-1) expression is suppressed by HS—Cf of the present invention. TNF-α-induced MMP-1 is one of the factors that damage and destroy cartilage. Mix HS—Cf in different concentrations with porcine chondrocytes and react for 2 hours. Then add TNF-α (5 ng/ml) to stimulate porcine chondrocytes for four hours. Next measure mRNA expression by real-time quantitative-polymerase chain reaction (Q-PCR). Thus it is confirmed that MMP-1 expression is decreased along with the increasing concentration of HS—Cf.

### Suppression of Matrix Metalloproteinase-3 (MMP-3)

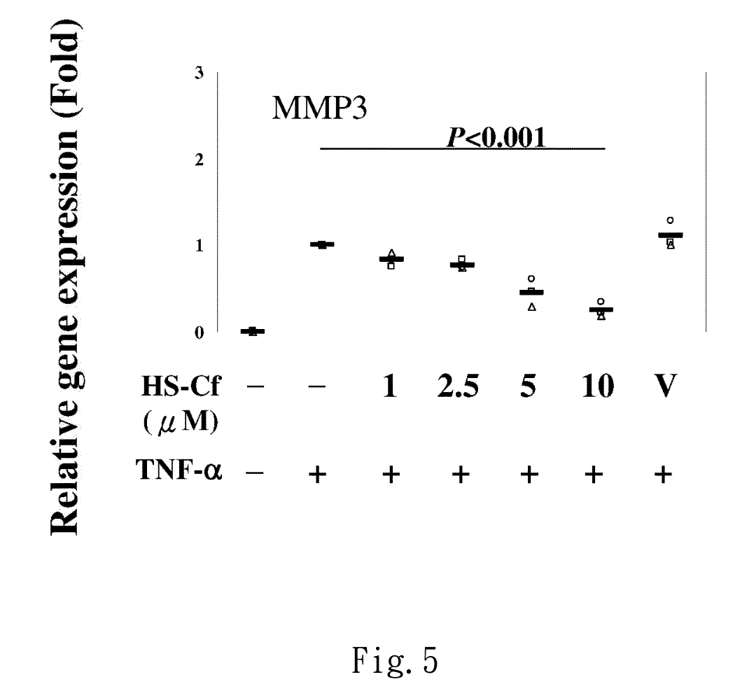

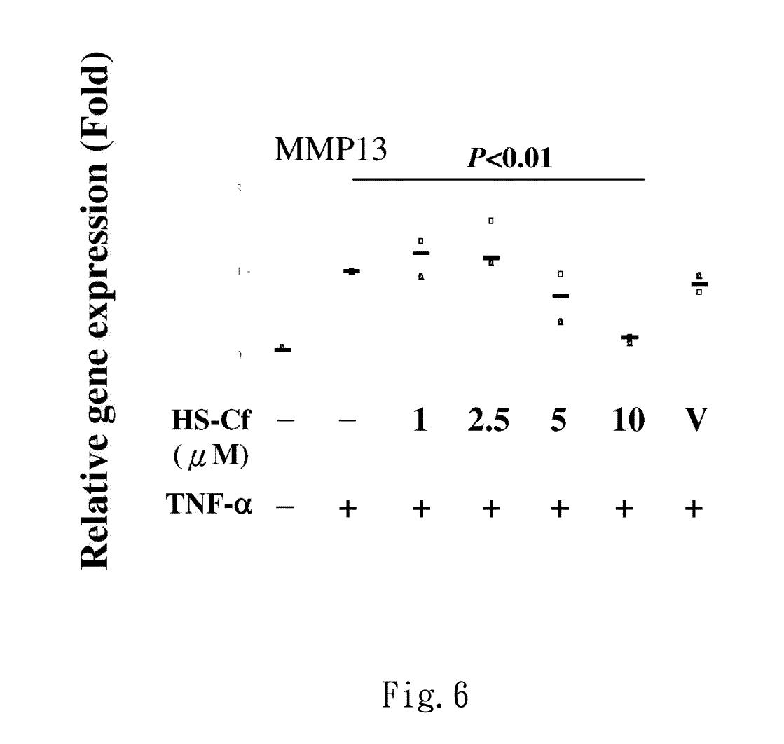

Refer to FIG. 5, it shows that matrix metalloproteinase-3 (MMP-3) expression is suppressed by HS—Cf of the present invention. TNF-α-induced MMP-3 is one of the factors that damage and destroy cartilage. Mix HS—Cf in different concentrations with porcine chondrocytes and react for 2 hours. Then add TNF-α (5 ng/ml) to stimulate porcine chondrocytes for four hours. Next measure mRNA expression by real-time quantitative-polymerase chain reaction (Q-PCR). Thus it is confirmed that HS—Cf decreases MMP-3 expression at higher concentration.

### Suppression of Matrix Metalloproteinase-13 (MMP-13)

Refer to FIG. 6, it shows that matrix metalloproteinase-13 (MMP-13) expression is suppressed by one of the factors that damage and destroy cartilage. Mix HS—Cf in different concentrations with porcine chondrocytes and react for 2 hours. Then add TNF-α (5 ng/ml) to stimulate cells for four hours. Next observe mRNA expression by real-time quantitative-polymerase chain reaction (Q-PCR). Thus it is confirmed that MMP-13 expression is decreased along with the increasing concentration of HS—Cf.

### Suppression of Aggrecanase-1

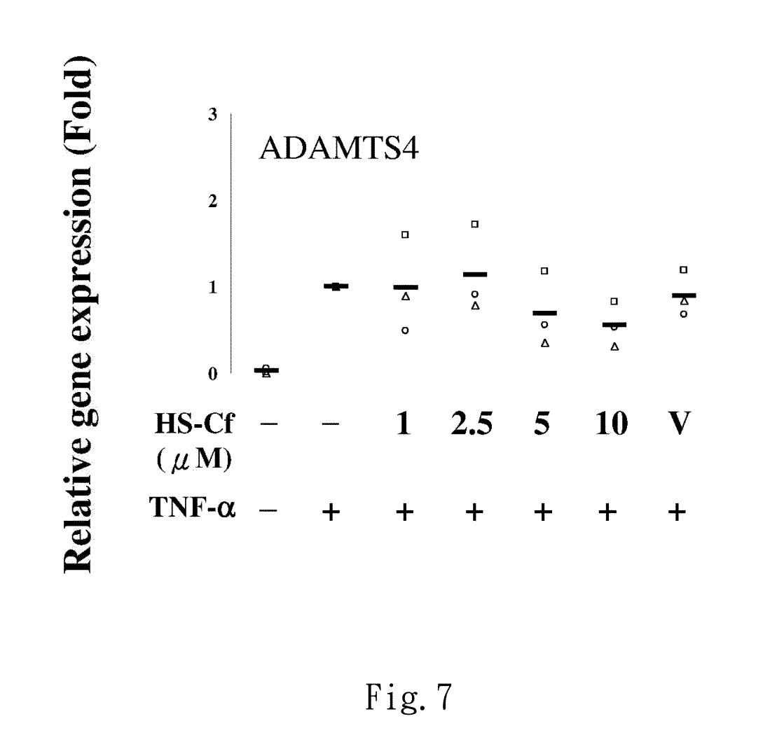

Refer to FIG. 7, it shows that A Disintegrin and Metalloproteinase with Thrombospondin motifs-4 (ADAMTS-4, also called Aggrecanase-1) expression is suppressed by HS—Cf of the present invention. TNF-α induced ADAMTS-4 is one of the factors that damage and destroy cartilage. Mix HS—Cf in different concentrations with porcine chondrocytes and react for 2 hours. Then add TNF-α (5 ng/ml) to stimulate cells for four hours. Next measure mRNA expression by Q-PCR. Thus it is confirmed that ADAMTS-4 expression is decreased along with the increasing concentration of HS—Cf.

### Suppression of Aggrecanase-2

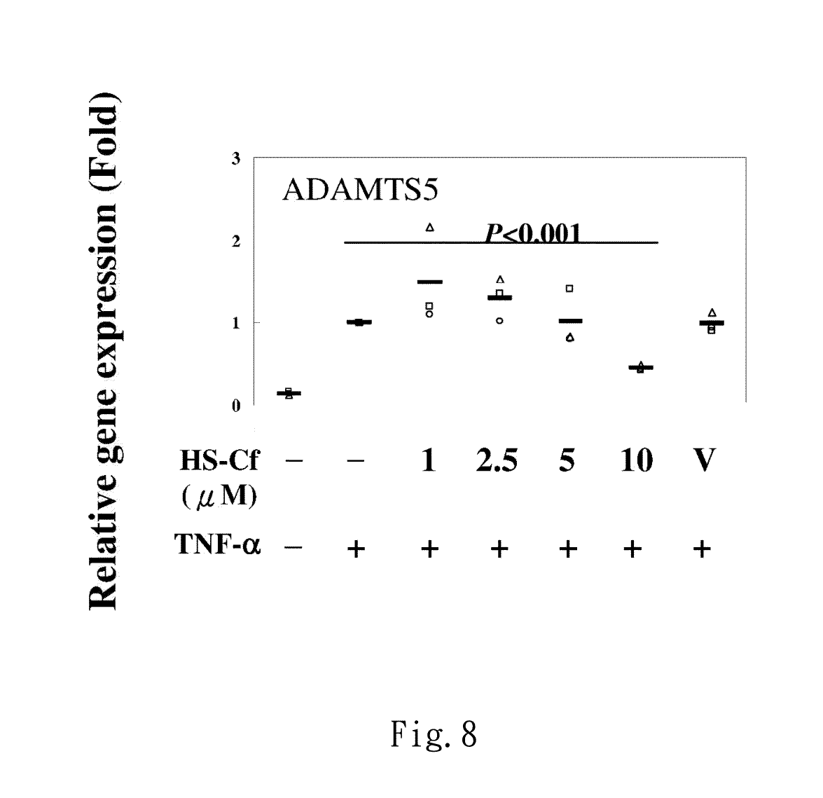

Refer to FIG. 8, it shows that A Disintegrin and Metalloproteinase with Thrombospondin motifs-5 (ADAMTS-5, also called Aggrecanase-2) expression is suppressed by HS—Cf of the present invention. TNF-α induced ADAMTS-5 is one of the factors that damage and destroy cartilage. Mix HS—Cf in different concentrations with porcine chondrocytes and react for 2 hours. Then add TNF-α (5 ng/ml) to stimulate cells for four hours. Next observe mRNA expression by Q-PCR. Thus it is confirmed that ADAMTS-5 expression is decreased along with the increasing concentration of HS—Cf.

### Loss Prevention of Collagen II

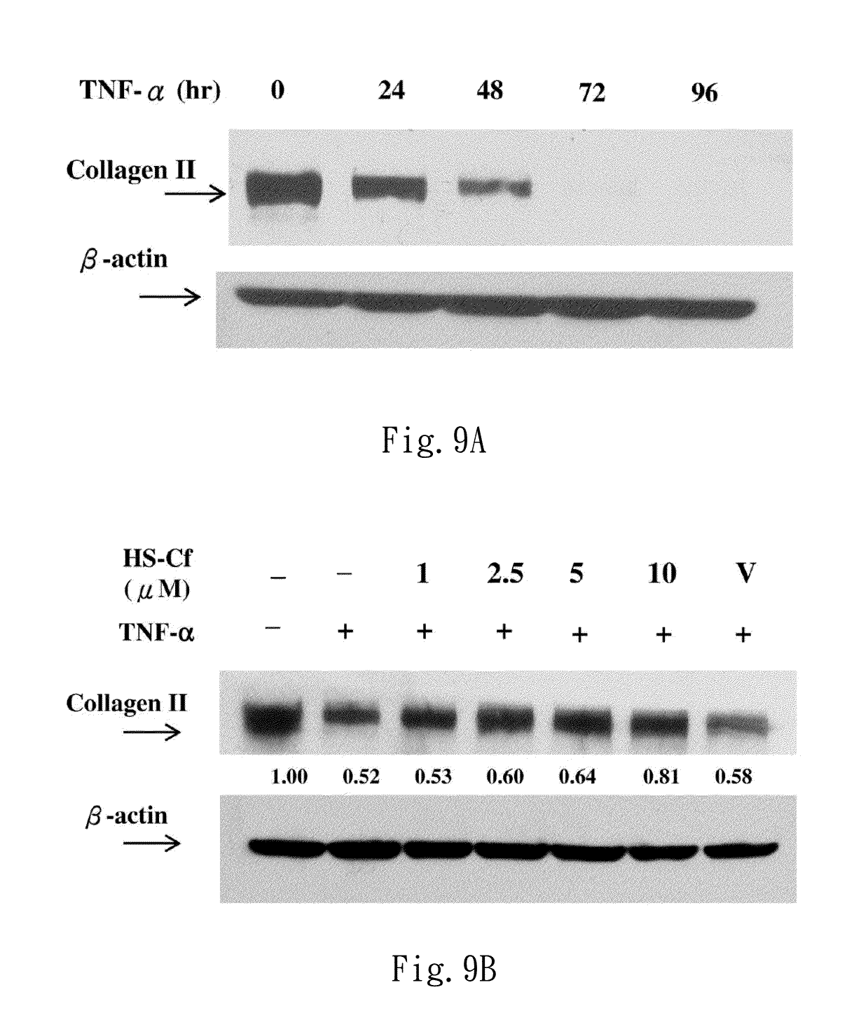

Refer to FIG. 9A, it shows loss of collagen II after TNF-α stimulation in the absence of HS—Cf of the present invention. As shown in the figure, after mixing porcine chondrocytes with TNF-α (5 ng/ml), the amount of collagen II at different times is determined by Western Blot. It is confirmed that collagen II is degraded and lost along with the time under TNF-α stimulation. However, refer to FIG. 9B, it shows the effect of the present invention on loss prevention of collagen II. As shown in figure, mix HS—Cf in different concentrations with porcine chondrocytes and react for 24 hours. Then add TNF-α (5 ng/ml) to stimulate porcine chondrocytes for 48 hours. Next use western blot to measure the amount of protein. The results show that the degradation and loss of collagen II is under control.

### Loss Prevention of Proteoglycan

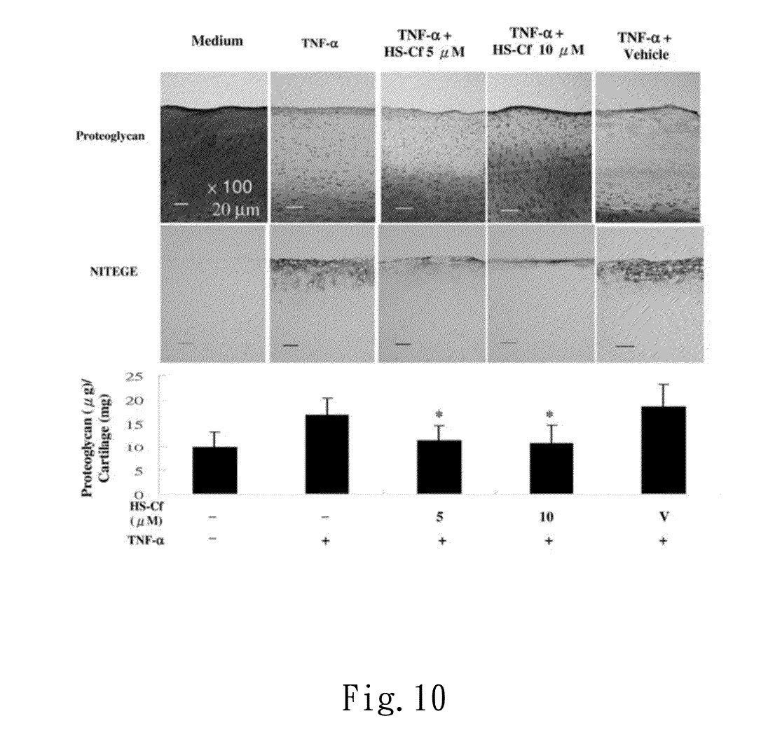

Besides collagen II, loss of proteoglycan (aggrecan) is also affected by HS—Cf. Refer to FIG. 10, it shows the effect of HS—Cf on the loss of proteoglycan. As shown in figure, mix HS—Cf in different concentrations with porcine chondrocytes and react for 24 hours. Then add TNF-α (5 ng/ml) to stimulate porcine chondrocytes for 72 hours. Next use Safranin-O staining to observe the amount of proteoglycan in cartilaginous tissue. Compared with results of TNF-α stimulation and 5/or 10 μM HS—Cf, it is learned that degradation and loss of proteoglycan is under control in the presence of HS—Cf.

Cytotoxicity of HS—Cf also has been checked. HS—Cf is used as a pharmaceutical composition so that cytotoxicity of HS—Cf has to be carefully considered for determining whether HS—Cf can be used for treatment of osteoarthritis. Through MTT (3-(4,5-Dimethylthiazol-2-yl)-2,5-diphenyltetrazolium bromide) cell viability assay and lactate dehydrogenase (LDH) assay, no obvious cytotoxic effect on porcine chondrocytes was detected at any of the concentrations. Thus HS—Cf is used in treatment of organisms.

By low bio-toxic HS—Cf that suppresses activity of factors involved in signaling pathways of TNF-α-induced IRF-1 expression such as iNOS, MMP-1, MMP-3, MMP-13, ADAMTS-4, ADAMTS-5, etc. with undetectable cytotoxicity, chondrocyte activation is suppressed and cartilage destruction is slowed down. At the same time, collagen II and proteoglycan are retained for maintaining functions of articular cartilage.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalent.

What is claimed is:

1. A pharmaceutical composition for treatment of osteoarthritis comprising a compound whose chemical structure is: 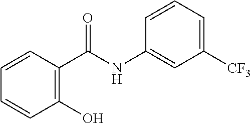 [SMILES: O=C(Nc1cccc(C(F)(F)F)c1)c1ccccc1O]
Wherein the pharmaceutical composition suppresses tumor necrosis factor (TNF) induced interferon regulatory factor (IRF).

2. The pharmaceutical composition as claimed in claim 1, wherein the tumor necrosis factor (TNF) is TNF-α.

3. The pharmaceutical composition as claimed in claim 1, wherein the interferon regulatory factor is interferon regulatory factor-1 (IRF-1).

4. The pharmaceutical composition as claimed in claim 3, wherein the interferon regulatory factor-1 activates matrix metalloproteinases, inducible nitric oxide synthase (iNOS), and aggrecanases.

5. The pharmaceutical composition as claimed in claim 4, wherein the matrix metalloproteinases include matrix metalloproteinase-1, matrix metalloproteinase-3 and matrix metalloproteinase-13.

6. The pharmaceutical composition as claimed in claim 4, wherein the aggrecanases include aggrecanase-1 (A Disintegrin and Metalloproteinase with Thrombospondin motifs-4, ADAMTS-4) and aggrecanase-2 (A Disintegrin and Metalloproteinase with Thrombospondin motifs-5, ADAMTS-5).

7. The pharmaceutical composition as claimed in claim 1, wherein the pharmaceutical composition further prevents TNF (tumor necrosis factor)-mediated collagen loss.

8. The pharmaceutical composition as claimed in claim 7, wherein the collagen is type II collagen.

9. The pharmaceutical composition as claimed in claim 1, wherein the pharmaceutical composition further prevents TNF (tumor necrosis factor)-mediated proteoglycan (aggrecan) loss.
